(19) United States
(12) Patent Application Publication
(10) Pub. No.: US 2022/0409664 A1
(43) Pub. Date: Dec. 29, 2022

(54) METHODS AND COMPOSITIONS FOR USE AS A PRE-TREATMENT FOR HIV THERAPIES

(71) Applicant: Regents of the University of Minnesota, Minneapolis, MN (US)

(72) Inventors: Pamela J. Skinner, Minneapolis, MN (US); Mary S. Pampusch, Minneapolis, MN (US)

(21) Appl. No.: 17/809,742

(22) Filed: Jun. 29, 2022

Related U.S. Application Data
(60) Provisional application No. 63/216,260, filed on Jun. 29, 2021.

(51) Int. Cl. A61K 35/17; C07K 16/28; A61K 9/00; A61P 31/18

(52) U.S. Cl. CPC A61K 35/17; C07K 16/2803; A61K 9/0019; A61P 31/18; A61K 2039/505

(57) ABSTRACT

Methods and compositions are described that can be used as a pre-treatment for HIV therapies.

### CROSS REFERENCE TO RELATED APPLICATIONS

This application claims benefit under 35 U.S.C. § 119(e) to U.S. Application No. 63/216,260 filed Jun. 29, 2021.

### FEDERALLY SPONSORED RESEARCH OR DEVELOPMENT

This invention was made with government support under AI143380 and AI096966-06S1 awarded by National Institutes of Health (NIH). The government has certain rights in the invention.

### TECHNICAL FIELD

This disclosure generally relates to methods and compositions for use as a pre-treatment for HIV therapies.

### BACKGROUND

Over 36 million individuals worldwide are currently living with HIV-1. Although antiretroviral therapy (ART) has produced substantial reductions in HIV-1-related morbidity and mortality, ART is costly and requires lifelong adherence to be effective. A major barrier to curing HIV infection is the persistence of viral replication, even in the presence of ART. Thus, developing strategies to induce sustained viral remission in the absence of ART is a priority for HIV-related research. The present disclosure describes methods and compositions that can be used to achieve this goal.

### SUMMARY

This disclosure describes methods and compositions that can be used as a pre-treatment for HIV therapies.

In one aspect, methods of temporarily depleting CD20+ cells in an individual being treated for HIV are provided. Such methods typically include administering CD20+ cell-depleting therapy to the individual for a period of time prior to and/or while the individual is receiving anti-HIV therapy, thereby temporarily depleting CD20+ cells in the individual.

In some embodiments, the CD20+ cell-depleting therapy comprises an anti-CD20 antibody (e.g., Rituximab or another CD20-depleting antibody).

In some embodiments, the CD20+ cell-depleting therapy is administered intravenously or intraparentally. Typically, administering the anti-CD20 antibody disrupts B cell follicles.

In some embodiments, the CD20+ cell-depleting therapy is administered at a dose of about 3 mg/kg recipient body weight up to about 9 mg/kg recipient body weight.

In some embodiments, the period of time is from about 1 day to about 4 weeks (e.g., about 3 days to about 25 days; about 5 days to about 3 weeks; about one week to about two weeks; about 10 days) prior to the individual receiving anti-HIV therapy.

In some embodiments, the anti-HIV therapy is selected from immunotherapy (e.g., anti-HIV immunotherapy), “shock and kill” therapy strategies, and anti-viral therapy (AVT). In some embodiments, the anti-HIV therapy is cell therapy (e.g., stem cell therapy). In some embodiments, the anti-HIV immunotherapy is an NK cell therapy. In some embodiments, the anti-HIV immunotherapy is autologous T cell therapy. In some embodiments, the anti-HIV immunotherapy is allogeneic T cell therapy.

In some embodiments, the anti-HIV immunotherapy comprises administering CD4-MBL-CAR/CXCR5 T cell therapy. In some embodiments, the CD4-MBL-CAR/CXCR5 T cell therapy is administered after the administration of the anti-CD20 antibody.

In some embodiments, such methods further include administering the anti-HIV therapy.

In some embodiments, the CD20+ cells that are temporarily depleted are mature B cells. Generally, the mature B cells are located within B cell follicles.

In another aspect, methods of preconditioning an individual for anti-HIV therapy are provided. Such methods typically include administering CD20+ cell-depleting therapy to the individual for a period of time prior to the individual receiving the anti-HIV therapy.

In some embodiments, the CD20+ cell-depleting therapy comprises an anti-CD20 antibody (e.g., Rituximab or another CD20-depleting antibody).

In some embodiments, the CD20+ cell-depleting therapy is administered intravenously or intraparentally. Typically, administering the anti-CD20 antibody disrupts B cell follicles.

In some embodiments, the CD20+ cell-depleting therapy is administered at a dose of about 3 mg/kg recipient body weight up to about 9 mg/kg recipient body weight.

In some embodiments, the period of time is from about 1 day to about 4 weeks (e.g., about 3 days to about 25 days; about 5 days to about 3 weeks; about one week to about two weeks; about 10 days) prior to the individual receiving anti-HIV therapy.

In some embodiments, the anti-HIV therapy is selected from immunotherapy (e.g., anti-HIV immunotherapy), “shock and kill” therapy strategies, and anti-viral therapy (AVT). In some embodiments, the anti-HIV therapy is cell therapy (e.g., stem cell therapy). In some embodiments, the anti-HIV immunotherapy is an NK cell therapy. In some embodiments, the anti-HIV immunotherapy is autologous T cell therapy. In some embodiments, the anti-HIV immunotherapy is allogeneic T cell therapy. In some embodiments, the anti-HIV immunotherapy comprises administering CD4-MBL-CAR/CXCR5 T cell therapy. In some embodiments, the CD4-MBL-CAR/CXCR5 T cell therapy is administered after the administration of the anti-CD20 antibody.

In some embodiments, such methods further include administering the anti-HIV therapy. In some embodiments, the CD20+ cells that are temporarily depleted are mature B cells. Generally, the mature B cells are located within B cell follicles.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The patent or application file contains at least one drawing executed in color. Copies of this patent or patent application publication with color drawings will be provided by the Office upon request and payment of the necessary fee.

FIG. 1A-1B are images showing CAR/CXCR5 T-cells home to lymphoid follicles and recognize SIV-infected cells in vivo. Location of cell trace violet dye (CTV)-labeled CAR/CXCR5 T-cells was determined in a chronically SIV-infected ART-naïve rhesus macaque, animal R14025 (T0) at 2 days post-treatment (DPT). FIG. 1A are representative images from spleen, lymph node (LN), bone marrow, ileum, lung, liver, brain, and rectum. Infused cells were labeled with CTV (pseudo-colored red). Tissues were stained with anti-IgM or anti-CD20 (green) to label B cells and delineate B cell follicles (green). Arrowheads point to CTV-labeled cells. Scale bars=100 μm. FIG. 1B show a representative image of spleen tissue section from animal R14025 (T0) showing duplex detection of CAR/CXCR5 construct (red) and SIV (pseudo-colored white) using RNAscope ISH combined with immunofluorescence using a custom-made probe for detection of CAR/CXCR5 construct and a probe specific for SIV. The white haze within the B cell follicle represents SIV virions trapped by the follicular dendritic cells (FDC) network. Scale bar=100 μm. The right panels of panel (1B) are enlargements showing an interaction between two CAR/CXCR5-transduced T cells and an SIV-infected cell. The tissue was stained with DAPI (blue), and anti-CD20 (green) to label B cells and delineate B cell follicles. Scale bar=10 μm. Confocal images were collected using a 20× objective. The curves tool in Photoshop was used to increase the contrast of each image in a similar manner.

FIG. 2 is a schematic showing a timeline of the rhesus macaque pilot studies. Animals were infected with SIV mac251 and ART suppressed. Cells were collected for transduction either post-infection (T1) or pre-infection (T2). ART was interrupted at the time of CAR/CXCR5 cell infusion. Blood and tissue samples were collected at regular intervals post-infusion.

FIG. 3A-3E are graphs showing post-infusion viral loads. Viral loads over time in untreated control animals (FIG. 3A), pilot study T1 animals (FIG. 3B) and pilot study T2 animals (FIG. 3C). Viral loads were determined by measurement of gag mRNA relative to the housekeeping gene beta-actin in cell pellets using reverse transcription (RT) polymerase chain reaction (PCR). Viral loads for the three animal groups one month post-infusion (27-30 DPI) (FIG. 3D). The bar represents the median. Levels of tetramer⁺ CD3 T cells at 28 days post-infusion in peripheral blood mononuclear cells (PBMCs) of the three animal groups as determined by flow cytometry (FIG. 3E). The bar represents the median.

FIG. 4A-4D are images showing that CAR/CXCR5 T-cells continue to expand in vivo. CAR/CXCR5 T-cells expanded at the edges of B cell follicles in vivo at 2 DPT and accumulated within B cell follicles at 6 DPT. FIG. 4A are representative images from LN tissue from Rh2850 stained 2 DPT showing CAR/CXCR5 T-cell proliferation in the extrafollicular (EF) area near the follicular (F) zone. Tissues were stained with anti-CD3 (blue) to label T cells and anti-CD20 (green) to label B cells and delineate B cell follicles (F). CAR/CXCR5-infused cells were labeled with CTV (pseudo-colored red). Cells within F showed lower fluorescence intensity indicating division. Scale bar=50 μm. FIG. 4B shows representative images of LN tissue section from Rh2858 visualized using RNAScope ISH combined with IF, showing expansion of CAR/CXCR5T-cells at the edge of follicles at 2 DPT. The right panel is an enlargement of the left panel, and shows a cluster of CAR/CXCR5 T-cells (red) that appear to be expanding at the edge of the follicle. Tissues were stained with DAPI (blue) and anti-IgM (green) to label B cells and delineate B cell follicles (F), with the more brightly stained germinal center in the center of the F. FIG. 4C is a representative image of LN tissue from Rh2850, showing CAR/CXCR5 T-cell (red) proliferation in F and EF at 6 DPT detected by RNAScope ISH combined with IF. Tissues were stained with anti-IgM (blue) and anti-Ki67 (green) to mark activation and proliferation. B cell follicles are delineated with white lines. Scale bars=100 μm. Confocal images were collected using a 20× objective. FIG. 4D is a graph showing the percentage of CAR/CXCR5 T cells that were Ki67+ in follicular areas (F) and extrafollicular areas (EF) of lymph nodes. The animal demonstrating the greatest control (Rh2850) showed the highest percentage of F Ki67+ CAR/CXCR5 T cells (64%) and the animal that lost control (Rh2853) showed the lowest percentage of follicular Ki67+ CAR/CXCR5 T cells (9%).

FIG. 5A-5F are images and graphs showing that CAR/CXCR5 T-cells localize to over 90% of the follicles and persist for up to 28 days. CAR/CXCR5 T-cells successfully homed to over 90% of the B cell follicles at 6 DPT and persisted for up to 28 DPT in SIV-infected ART-suppressed/released animals. Representative images of LN tissue section from Rh2858, showing CAR/CXCR5 T-cells (red) detected using RNAScope ISH combined with IF using a custom-made probe for detection of the CAR/CXCR5 construct. FIG. 5A shows a confocal image showing a whole LN tissue section. FIG. 5B shows an enlargement of the delineated area in FIG. 4A, showing that CAR/CXCR5-transduced T cells (red) successfully homed to a B cell follicle (green). The tissue was stained with DAPI (blue), and anti-CD20 (green) to label B cells and delineate B cell follicles. The confocal image is collected with a 20× objective. Scale bar=100 μm. FIG. 5C shows levels of CAR/CXCR5 T-cells over time after infusion in follicular (green) and extrafollicular areas (blue) of LN. The animal that lost control of the virus infection is marked with a red star. Samples not determined are marked ND. FIG. 5D shows the percentage of follicles that contained CAR/CXCR5 T-cells over time post-infusion. Frequency of CD4-MBL⁺ cells in CD3⁺ PBMCs as determined by flow cytometry (FIG. 5E) and copies of CAR in the total cell population in BAL (FIG. 5F) as determined by quantitative real-time PCR at the indicated time points post-infusion.

FIG. 6A-6C show that infusion of CAR/CXCR5 T-cells into SIV-infected rhesus macaques results in lower viral loads post-ART interruption. At 28 DPT, treated animals showed a reduction in viral (v)RNA compared to untreated control animals. FIG. 6A shows representative images from a LN tissue section showing the abundance of SIV vRNA⁺ cells and free virions trapped by follicular dendritic cells network (pseudo-colored white) detected using RNAscope ISH combined with IF in treated from Rh2850 (left panels) versus untreated control from R11002 (right panels). The tissue was stained with DAPI (blue), and anti-CD20 (green) to label B cells and delineate B cell follicles. The white haze within the B cell follicle represents SIV virions trapped by the follicular dendritic cells (FDC) network. Confocal images were collected with a 20× objective. Scale bar=100 μm. (FIG. 6B) Levels of viral RNA in F and EF areas. The animal that lost viral control of the virus infection is marked with a red star. FIG. 6B shows that the two treated animals that exhibited sustained control of SIV infection (Rh2850, Rh2858) showed few to no SIV vRNA+ cells at 28 days post-treatment in F and EF areas compared to abundant SIV vRNA+ cells in untreated control animals and in the T2 animal that did not control the infection (Rh2853). FIG. 6C shows the percentage of follicles with free virions bound by FDC.

FIG. 7A-7F shows that immunotherapeutic cell infusion leads to transient increases in cytokine levels and cell accumulation in bronchoalveolar lavage fluid. Serum samples were analyzed for post-infusion production of cytokines using a non-human primate (NHP)-specific tumor necrosis factor (TNF) alpha (FIG. 7A), interferon (IFN) gamma (FIG. 7B), interleukin (IL)-6 (FIG. 7C) and IL-2 multiplex Luminex assay (FIG. 7D). Lung accumulation of CAR T-cells was determined by analysis of bronchoalveolar lavage (BAL) samples. Cells were isolated from BAL and analyzed for the percentage of CD4-MBL CAR⁺ cells in the CD3⁺ T population using flow cytometry (FIG. 7E) or the number of copies of CAR/cell in the total cell population using quantitative real-time PCR (FIG. 7F).

FIG. 8 shows representative flow plots from cells prepared for infusion. 1×10⁶ cells were stained with the antibodies listed in the Flow Cytometry section of the Methods. Gating strategy for determination of co-expression of CAR (MBL) and CXCR5. The CD8+ population was used to determine central memory phenotype (CD28⁺CD95⁺) and CCR7 expression. Plots presented are from cells infused into Rh2858.

FIG. 9A-9C shows phenotypic analysis of CAR/CXCR5 transduced cells. 1×10⁶ cells were stained for flow cytometry with the antibodies listed in the flow cytometry section. Plots represent activation and proliferation of expanded cells as measured by Ki67 and CD25 expression (FIG. 9A), expression of inhibitory markers, PD-1 and CTLA-4 (FIG. 9B), and the level of apoptosis as determined by annexin staining (FIG. 9C).

FIG. 10 is a graph showing the numbers of follicular CAR/CXCR5 T-cells identified in situ in lymph nodes correlate with numbers of CAR-specific PBMCs identified by flow cytometry. Correlation between follicular CAR/CXCR5 T-cells/mm² by RNAScope and CD4-MBL⁺ PBMC by flow cytometry. Association was tested using Spearman's correlation, assuming independence. Scales are log (value+1) on the y-axis and log (value) on the x-axis; labels use the original units. The line represents the fitted regression.

FIG. 11 is a schematic showing an exemplary experimental outline. Briefly, Mamu-A*001:01 MHC-I allele positive and negative rhesus macaques are selected at the Wisconsin National Primate Research Center (WNPRC). Animals that possess MHC-I alleles associated with spontaneous control of viremia (e.g., B*08 or B*17) are excluded from the study. For certain studies, PBMCs are collected, prior to infection with SIVmac239, and transduced with CAR/CXCR5 (n=6). Transduced cells are infused at the WNPRC. Control animals do not receive infused cells (n=6). Animals are infected with SIVmac 239, cART suppressed from day 35-45 after SIV challenge, and 1 week prior to cell infusion treated with CD20 antibodies. Approximately 2×10\hat{ }8 cells/kg are infused, and animals are released from cART. Collection of blood, BAL, LN biopsies, GALT biopsies and necropsy are indicated by arrows.

FIG. 12A-12B shows that anti-CD20 antibody treatment led to a reduction of B cells in lymphoid tissues 9 days post-treatment. Spleen (FIG. 12A) and lymph node (FIG. 12B) from animal R09072 collected at necropsy 2 days post-CAR/CXCR5 infusion and 9 days post-anti-CD20 antibody treatment were stained with anti-CD20 antibodies (green) to detect B cells and delineate follicles. Sections were counter-stained with DAPI (blue) to identify cell nuclei. The images on the right are magnified versions of the boxed regions shown in the images on the left.

FIGS. 13A-13B shows that anti-CD20 antibody led to a temporary reduction of B cells in blood. SIV-infected, ART-suppressed rhesus macaques were treated with 7 mg/kg Rituximab seven days prior to ART release and the infusion of CAR/CXCR5 T cells. FIG. 13A shows representative flow cytometry plots of CD20 stained B cells from animal Rh2997 at 7 and 35 days after anti-CD20 antibody treatment. For these plots, live CD20+ cells were gated from the total lymphocyte population. FIG. 13B shows the percentage of lymphocytes in the blood that were CD20+ in anti-CD20 antibody-treated animals over time. These included 3 CAR/CXCR5 T cell treated animals (blue) and 4 animals that did not receive CAR/CXCR5 T cells (red).

FIG. 14 shows the expansion of CAR T cells in SIV-infected, ART-suppressed rhesus macaques treated with anti-CD20 antibody seven days prior to ART release and infusion of CAR/CXCR5 T cells. Doses of CAR/CXCR5 T cells infused are indicated at the bottom of the figure. The concentration of CAR/CXCR5 T cells and SIV vRNA+ was determined at days 2 and 6 post-infusion, in lymph nodes in situ using RNAscope analysis. No vRNA+ cells were detected. Levels of CAR/CXCR5 T cells are indicated for three animals (rh3027, rh2997, rh2783). The fold-change in concentration of CAR/CXCR5 T cells for each animal is indicated above each bar graph.

FIG. 15 shows that CAR/CXCR5 cells accumulated to extremely high levels in follicles. Representative image (left panel) and magnified image (right panel) of a lymph node section from animal R2997 at 6 days post infusion. RNAScope was used to detect SIV (white) and CAR/CXCR5 (red). RNA and immunofluorescence was used to detect B cells and delineate the follicles (anti-CD20, green—delineated with a line on the right). Sections were counter-stained with DAPI (blue), which stains cell nuclei. No vRNA cells were detected. CAR/CXCR5 T cells accumulated to extremely high levels, preferentially in follicles. Scale bars 1000 μm left and 50 μm right.

### DETAILED DESCRIPTION

This disclosure describes methods and compositions for the temporary depletion of B cells as a non-toxic preconditioning strategy for anti-HIV therapy. Without being bound by any particular theory, such a pre-conditioning strategy may temporarily disrupt and expose the follicular reservoir of HIV virus to therapeutic cells, and simultaneously promote engraftment of therapeutic cells.

During chronic HIV or simian immunodeficiency virus (SIV) infection prior to AIDS, viral replication concentrates within the B-cell follicle, primarily within the T follicular helper cells (Tfh). Free virions in immune complexes are also localized to the follicle through binding to follicular dendritic cells (FDC) in the germinal centers (GC). By contrast, levels of virus-specific CD8⁺ T-cells, which are critical in controlling HIV or SIV infection, are found at relatively low levels within B-cell follicles. In fact, we previously reported the average ratio of in vivo effector (virus-specific CD8⁺ T-cells) to target (virus-infected cells) cells is 40-fold lower in follicular (F) compartments compared to extra-follicular (EF) compartments of secondary lymphoid tissues (SLT). Further, we reported that levels of SIV in F and EF areas of SLT are inversely correlated with levels of virus-specific CD8⁺ T-cells in the same areas. Migration of lymphocytes into B-cell follicles is directed by the binding of the chemokine receptor, CXCR5, to the chemokine ligand, CXCL13, which is produced by follicular stromal cells, such as marginal reticular cells and FDC, and by GC Tfh cells. Thus, expression of CXCR5 on the surface of a CD8⁺ T-cell mediates migration into B-cell follicles.

Anti-CD20 monoclonal antibodies (mAbs) were initially developed to treat B cell proliferative disorders including non-Hodgkin's lymphoma (NHL) and chronic lymphocytic leukemia (CLL). Anti-CD20 mAbs have subsequently been tested and used in the treatment of autoimmune disorders such as rheumatoid arthritis (RA), systemic lupus erythematosus (SLE) and multiple sclerosis (MS). Representative anti-CD20 antibodies include, without limitation, Rituximab (and variants thereof, e.g., Rituximab-hyaluronidase), Ofatumumab, Obinutuzumab, Ibritumomab tiuxetan, and Ibritumomab tiuxetan. To date, anti-CD20 antibodies have not been used in a pre-treatment regimen for anti-HIV therapies.

The dose regimen described herein for the anti-CD20 antibody is very different from the dose regimens that have been used to date for anti-CD20 antibodies in the clinic. Specifically, the CD20 depletion described herein can be very short-lived (e.g., temporary), and is primarily relevant during the time an individual is receiving anti-HIV or other therapy. For example, an anti-CD20 antibody can be administered to an individual coincidentally with the delivery of anti-HIV therapy or an anti-CD20 antibody can be administered to an individual in advance of the individual receiving anti-HIV or other therapy (e.g., about 1 day, 2 days, 5 days, 7 days, 10 days, or 14 days prior to the individual receiving anti-HIV or other therapy). In some instances, it may be desirable to continue delivering an anti-CD20 antibody after anti-HIV therapy has ceased (e.g., for 12, 24 or 48 hours after anti-HIV therapy has ceased).

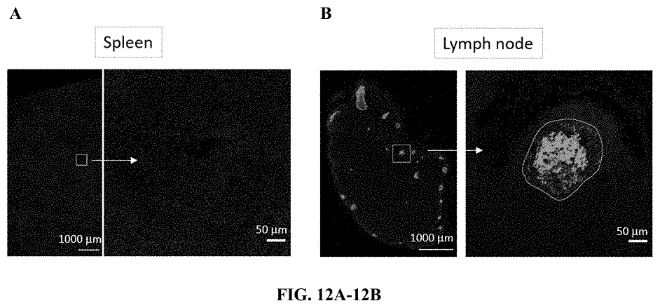

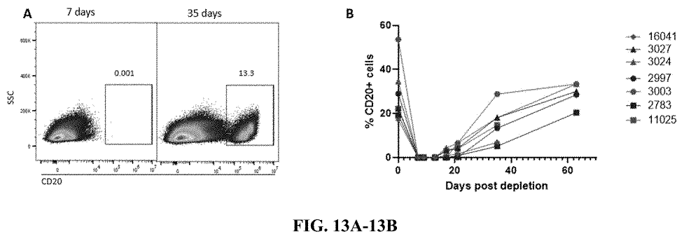

A dose of about 7 mg/kg of recipient body weight of CD20-depleting antibodies in rhesus led to the temporary depletion of B cells in lymph nodes and spleen (FIG. 12) and in the blood (FIG. 13B). B cells returned to normal levels in blood by 60 days post-treatment (FIG. 13B). Therefore, a dose of about 3 mg/kg of recipient body weight to about 9 mg/kg of recipient body weight (e.g., about 3 mg/kg of recipient body weight to about 7 mg/kg of recipient body weight; about 5 mg/kg of recipient body weight to about 8 mg/kg of recipient body weight; about 5 mg/kg of recipient body weight; or about 7 mg/kg of recipient body weight) can be used as described herein for pre-conditioning. The dose described herein is lower than that prescribed for other diseases presently treated with anti-CD20 depleting antibodies. Current therapies using anti-CD20 antibodies use doses that are much higher (see, e.g., Table 1) than the doses described herein and are associated with much longer depletion of B cells than are described herein.

For example, the prescribed dose of anti-CD20 antibodies (e.g., Rituximab) for treating cancer and autoimmune diseases ranges from about 9.6 mg/kg of recipient body weight per dose up to about 13.5 mg/kg of recipient body weight per dose, and the prescribed number of those doses of anti-CD20 antibodies for treating cancer and autoimmune diseases ranges from at least twice to more than eight times (see Table 1). As demonstrated herein, a single dose treatment of a low dose of anti-CD20 antibodies (e.g., about 3 mg/kg of recipient body weight to about 9 mg/kg of recipient body weight) is sufficient to temporarily deplete B cells from the blood and secondary lymphoid tissues, while allowing B cell levels to return to original levels in a couple of weeks (e.g., 1 week, 2 weeks, 3 weeks, or 4 weeks) up to a couple of months (e.g., 1 month, 2 months, or 3 months).

TABLE 1
Current Anti-CD20 Antibody Doses

| | | | mg/kg | |
|---|---|---|---|---|
| | | | (assuming | |
| Disease | Dose | | 74 kg) | Dosage |
| Non-Hodgkins Lymphoma | 374 | mg/m2 | 9.62 | weekly |
| | | | | (4-8 doses) |
| Chronic Lymphocytic | 375-500 | mg/m2 | 9.62-12.8 | multiple |
| Leukemia | | | | |
| Arthritis | 1000 | mg | 13.5 | 2 doses |
| Microscopic Polyangiitis | 375 | mg/m2 | 9.62 | 4 doses |
| Multiple Sclerosis | 1000 | mg | 13.5 | 2 doses |
| Pemphigus Vulgaris (PV) | 1000 | mg | 13.5 | 2 doses |
| Wegener Granulomatosis | 375 | mg/m2 | 9.62 | 4 doses |

Anti-HIV therapies are known in the art, and include, without limitation, immunotherapy, “shock and kill” therapy strategies, and anti-viral drug therapy. CD4-MBL-CAR/CXCR5 T cell immunotherapy is known in the art and is described in, for example, US 2018/0371057; “shock and kill” therapies are known in the art and is described in, for example, Kuse et al., 2021, J. Virol., JV10069921; and anti-viral therapies are known in the art and are described in, for example, Arts & Hazuda, 2012, Cold Spring Harbor Perspect. Med., 2:a007161.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Animal Study Design

The studies used 10 rhesus macaques that were positive for the class I allele Mamu-A1*001 but negative for Mamu-B*008 and Mamu-B*017:01. Rhesus macaques were housed at the Wisconsin National Primate Research Center (WNPRC). All procedures were approved by the University of Wisconsin-Madison College of Letters and Sciences and Vice Chancellor for Research and Graduate Education Centers Institutional Animal Care and Use Committee (IACUC protocol number G005529). The animal facilities of the Wisconsin National Primate Research Center are licensed by the US Department of Agriculture and accredited by AAALAC.

Animals were monitored twice daily by veterinarians for any signs of disease, injury, or psychological abnormalities. At the conclusion of the study, animals were humanely euthanized. The initial treated animal (T0) was chronically infected with SIVmac239 for 20 months prior to treatment. T0 was necropsied at day 2 post-infusion in order to determine the abundance and localization of the infused cells. Pilot study 1 and 2 treated animals (T1 and T2) and control untreated animals (n=3 per group) were infected intrarectally with SIVmac251 (1×10⁸ viral RNA). ART consisting of 5.1 mg/kg Tenofovir Disoproxil Fumerate (TDF) (Gilead), 40 mg/kg Emtricitabine (FTC) (Gilead) and 2.5 mg/kg Dolutegravir (DTG) (Viiv) was formulated at Beth Israel Deaconess Medical Center (BIDMC). ART was initiated at day 63-68 post-infection and continued daily until the day of cell infusion. Blood samples were drawn biweekly to monitor viral loads and all animals had undetectable viral loads at the time of infusion. Animals were ART-suppressed for times indicated in Table 2. PBMC were collected by density gradient centrifugation from blood draws either post-infection for T1 or pre-infection for T2. PBMCs were cryopreserved in CryoStor CS5 (BioLife Solutions Inc.) at a concentration between 4 and 20 million cells/mL and transported and stored in liquid nitrogen until use.

### Example 2—Cell Manufacturing and Infusion

The CD4-MBL CAR/CXCR5 construct was described previously (Haran et al., 2018, Front. Immunol., 9:1-12). The bi-specific CAR contains rhesus CD4 and mannose-binding lectin (MBL) domains, which leads to specificity for simian immunodeficiency virus (SIV), linked to extracellular hinge, transmembrane and co-stimulatory domains of rhesus CD28 (Liu et al., 2015, J. Virol., 89:6685-94; Ghanem et al., 2018, Cytotherapy, 20:407-19). The follicular homing receptor, CXCR5, is linked to the CAR with a self-cleaving peptide, P2A. Gammaretroviruses were produced by lipofectamine-mediated transfection of 293T cells (Haran et al., 2018, supra). CD4-MBL CAR/CXCR5 T-cells were manufactured using the CD4-MBL CAR/CXCR5 gammaretrovirus as outlined previously (Pampusch et al., 2020, Mol., Ther.—Methods of Clin. Dev., 16:1-10; Pampusch & Skinner, 2020, J. Vis. Exp., 60400). Prior to infusion, cells were stained with Cell Trace Violet, an intracellular fluorescent dye, resuspended at a density of 2×10⁷ cells/mL in RPMI for T0 and in PBS containing 10% autologous serum for all other animals, packed on ice and transported to the WNPRC. The CAR/CXCR5 T-cells were infused intravenously over 20 min while the animals were sedated. A veterinarian was present during the entire infusion. The dose of cells ranged from 0.35 to 2×10⁸ cells/kg (Table 2). Following infusion, animals were evaluated for signs of pain, illness, and stress observing appetite, stool, typical behavior and physical condition by the staff of the Animal Services Unit at least twice daily. The weight of the animals was monitored routinely throughout the protocol.

### Example 3—Tissue, Blood and Cell Collection

Blood samples were drawn for viral load determination immediately before and after infusion and on days 2, 6, 10, 14 and then biweekly until necropsy. Complete blood counts (CBC) were monitored biweekly throughout the experiment. LN biopsies and BAL samples were collected on days 2, 6, 14, 28 and 60-69 post-infusion. Colon and rectal biopsies were collected on days 2, 14, 28 and 60-69 post-infusion. Animals were necropsied between day 69 and 302 post-infusion.

### Example 4—Viral Load Determination

Viral loads were measured by Virology Services (WNPRC). Viral RNA was isolated from plasma samples using the Maxwell Viral Total Nucleic Acid Purification kit on the Maxwell 48RSC instrument (Promega, Madison Wis.). Viral RNA was then quantified using a highly sensitive QRT-PCR assay based on the one described (Cline et al., 2005, J. Med. Primatol, 34:303-312). RNA was reverse transcribed and amplified using TaqMan Fast Virus 1-Step Master Mix qRT-PCR Master Mix (Invitrogen) on the LightCycler 480 or LC96 instrument (Roche, Indianapolis, Ind.) and quantified by interpolation onto a standard curve made up of serial tenfold dilutions of in vitro transcribed RNA. RNA for this standard curve was transcribed from the p239gag_Lifson plasmid kindly provided by Dr. Jeffrey Lifson, (NCI/Leidos). The final reaction mixtures contained 150 ng random primers (Promega, Madison, Wis.), 600 nM each primer and 100 nM probe. Primer and probe sequences are as follows: forward primer: 5′-GTC TGC GTC ATC TGG TGC ATT C (SEQ ID NO:1), reverse primer: 5′-CAC TAG CTG TCT CTG CAC TAT GTG TTT TG-3′ (SEQ ID NO:2) and probe: 5′-6-carboxyfluorescein-CTT CCT CAG TGT GTT TCA CTT TCT CTT CTG CG-BHQ1-3′ (SEQ ID NO:3). The reactions cycled with the following conditions: 50° C. for 5 min, 95° C. for 20 s followed by 50 cycles of 95° C. for 15 s and 62° C. for 1 min. The limit of detection of this assay is 100 copies/mL.

### Example 5—Flow Cytometry

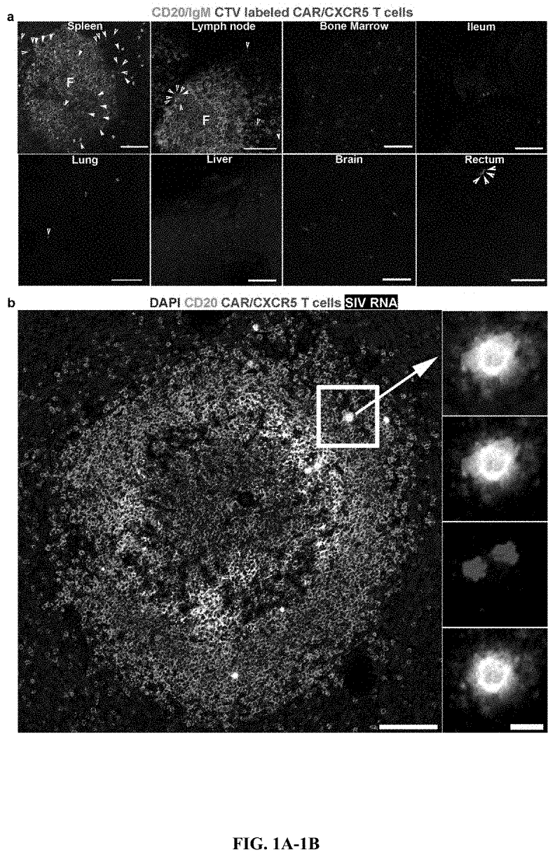

Multiparametric flow cytometry was performed on fresh, transduced PBMC and on thawed PBMC or BAL cells collected post-infusion with monoclonal antibodies cross-reactive in rhesus macaques to detect CD4-MBL-CAR/CXCR5 T-cells and SIV-specific T-cells. Cells were incubated with Live/Dead NIR (Invitrogen); Alexa Fluor 700 mouse anti-human CD3 (SP34-2), FITC, Brilliant Violet 650 mouse anti-human CD4 (M-T477), Brilliant Violet 510 mouse anti-human CD8 (RPA-T8), PerCP/Cy5.5 mouse anti-human CD95 (DX2) and Brilliant Violet 605 mouse anti-human CD28 (28.2) (BD Biosciences); Phycoerythrin (PE) mouse anti-human CXCR5 (MU5UBEE) (eBiosciences); MBL (3E7) (Invitrogen) conjugated to Alexa Fluor 647. To detect SIV-specific CD8⁺ T-cells, samples were incubated with PE-labeled GAG-CM9 (NIH Tetramer Core) at 37 C for 15 min. For phenotypic analysis, the following antibodies were used from BD: Brilliant Violet 650 mouse anti-human Ki67 (B56); from Biolegend: Brilliant Violet 785 mouse anti-human CD152 (CTLA-4) (BN113), Brilliant Violet 605 mouse anti-human Programmed cell death (PD-1) (EH12.2H7), Pacific Blue mouse anti-human Granzyme B (GB11); From Tonbo: PECyanine7 mouse anti-human CD25 (BC96); From MabTech: FITC mouse anti-human Perforin (Pf-344). To measure apoptosis, cells were washed 1× in apoptosis binding buffer, stained with Annexin V (BD) (1:20 dilution) and incubated for 20 minutes at room temperature. Cells were analyzed by flow cytometry within 2 hours of staining. All samples were acquired on LSRII (BD) or CytoFlex (Beckman Coulter). A minimum of 100,000 events were acquired for each sample. Data was analyzed with FlowJo v10 (Becton Dickinson and Company). Representative flow plots for transduced cells are presented in extended data FIG. 1.

### Example 6—CAR/CXCR5 T-Cell PCR

DNA qPCR was used to determine the quantity of CAR T-cells in PBMC and BAL. Genomic DNA was isolated from freshly thawed PBMC or BAL collected post-infusion using the DNeasy Blood and Tissue kit (Qiagen). PCR primers were designed to specifically bind to the junction of the CD4 and MBL fragments of the CAR in order to avoid recognition of endogenous CD4 or MBL. 300 nM concentrations of the following primers were used in the assay: CAR forward primer 5′-ATA TTG TGG TCC TGG CCT TTC A-3′ (SEQ ID NO:4); CAR reverse primer 5′-AAG AAT TTG TTT CCG ACC TGC C-3′ (SEQ ID NO:5); albumin forward primer 5′-TGC ATG AGA AAA CGC CAG TAA-3′ (SEQ ID NO:6); albumin reverse primer 5′-ATG GTC GCC TGT TCA CCA A-3′ (SEQ ID NO:7). PCR was run on a CFX96 thermal cycler (BioRad) with a program of one cycle of denaturation at 95° C. for 2 min, followed by 40 cycles of 95° C. for 10 sec and 60° C. for 30 sec. An amplified DNA fragment of the CAR was used in a standard curve in order to determine the copy number of the CAR. Albumin, which is present as two copies per cell, was used in order to determine cell number. The limit of detection was 2 copies of CAR DNA per 10⁵ cells.

### Example 7—Luminex Assay

Serum samples were stored at −80 C prior to analysis. Samples were tested by the Cytokine Reference Laboratory (University of Minnesota) using the magnetic bead set PRCYTOMAG-40K (EMD Millipore). Samples were analyzed for Non-Human Primate (NHP) specific TNFalpha, IFNgamma, IL-6 and IL-2 using the Luminex platform and done as a multi-plex. Fluorescent color-coded beads coated with a specific capture antibody were added to each sample. After incubation, and washing, biotinylated detection antibody was added followed by phycoerythrin-conjugated streptavidin. The beads were read on a Luminex instrument (Bioplex 200). Samples were run in duplicate and values were interpolated from five-parameter fitted standard curves.

### Example 8—Singleplex RNAScope In Situ Hybridization and Immunohistochemistry

RNAScope in situ hybridization utilized the 2.5 HD Reagent RED kit (Advanced Cell Diagnostics) as described previously (Deleage et al., 2016, Pathog. Immun., 1:68; Vasquez et al., 2018, J. Histochem. Cytochem., 66:427-46; Bertram et al., 2019, Nat. Commun. 10:1-15) with modifications. Five μm FFPE tissue sections on slides were deparaffinized by baking 1 h at 60° C., rinsing in xylene followed by absolute ethanol and air-drying. Sections were boiled in RNAScope® 1×Target Retrieval buffer (Advanced Cell Diagnostics) for epitope retrieval. Sections were then washed in dH₂O, dipped in absolute ethanol and air-dried. Following a protease pretreatment, sections were rinsed with dH₂O and hybridized overnight at 40° C. with one of the following probes (all from Advanced Cell Diagnostics); SIVmac239 no-env antisense probe or a custom-made probe for the gammaretroviral vector to detect the CAR/CXCR5-Transduced cells, DapB probe as a negative control probe or Macaca mulatta peptidylprolyl isomerase B (cyclophilin B) probe served as a positive control. Sections were then washed with 0.5×RNAScope wash buffer (Advanced Cell Diagnostics) and incubated with amplification reagents (1-6) according to the manufacturer's instructions. For chromogenic detection, sections were incubated with 120 μL of fast Red chromagen solution and washed as recommended by the manufacturer. For immunofluorescence staining, sections were blocked with 4% normal goat serum (NGS) and incubated overnight with the following primary antibodies: mouse-anti-human CD20 (Clone L26, Biocare), mouse-anti-CD68 (KP1; Biocare), rabbit anti-CD20 (Polyclonal, Thermo Scientific), rabbit anti-CD3 (SP7; Labvision/Thermo Scientific), rabbit anti-CD4 (EPR6855; Abcam), or rabbit anti-Ki67 (Clone SP6, Invitrogen/Thermo Scientific). Goat secondary antibodies (Jackson

Immunoresearch Laboratories) against mouse, rabbit, or human IgM conjugated to Alexa Fluor™ 488, Alexa Fluor™ 647 or Cy5 were used. Sections were counterstained with 1 μg/mL DAPI and mounted in Prolong® Gold (ThermoFisher Scientific).

### Example 9—Duplex In Situ Hybridization Combined with Immunofluorescence

For simultaneous visualization of both SIV vRNA and CAR/CXCR5-transduced cells, the RNAScope multiplex fluorescent kit V2 (Advanced Cell Diagnostics) was used with the opal fluorophores system (Akoya Bioscience) according to the manufacturer's instructions and as previously described (Vasquez et al., 2018, supra) with some modifications. In brief, 5 μm FFPE tissue sections on slides were deparaffinized as described above. Sections were pretreated with H₂O₂ (to block endogenous peroxidase activity) and washed in dH₂O. Heat-induced epitope retrieval was achieved by boiling sections in RNAScope® 1×target retrieval buffer (Advanced Cell Diagnostics). Sections were washed, dehydrated in absolute ethanol, and air-dried. Sections were incubated with protease solution, rinsed twice in d H₂O and incubated with pre-warmed premixed target probes (all from Advanced Cell Diagnostics) in which SIVmac239 no env antisense probe channel 2 (C2) was diluted in the custom-made probe for the gamma-retroviral vector to detect the CAR/CXCR5-transduced cells channel 1 (C1) at C2: C1 1:50 ratio overnight at 40° C. Sections were washed with a 0.5×RNAScope wash buffer. Amplification and HRP-C1 and HRP-C2 signal development were performed as recommended by the manufacturer with the modification of the use of 0.5×RNAScope wash buffer instead of 1×RNAScope wash buffer and use of a 1:150 dilution of Opals (all from Akoya Bioscience) instead of 1:1500. Opal™ 570 and Opal™ 690 were used for C1 and C2 respectively. For immunofluorescent staining, sections were washed twice in TBST (TBS—tween 20-0.05% v/v), blocked in 10% NGS—TBS-1% BSA and incubated with primary antibodies diluted in TBS-1% BSA for 1 h at room temperature (RT). Primary antibodies included the same antibodies described in Singleplex vRNA in situ hybridization combined with immunofluorescence. The sections were washed and incubated with secondary antibodies, Opal Polymer HRP Ms+Rb for 10 min at RT. After washing, the sections were incubated with Opal™ 520 diluted 1:150 in the multiplex TSA buffer (Advanced Cell Diagnostics) for 10 min at RT. After washing, sections were counterstained with 1 μg/mL DAPI and mounted in Prolong® Gold (ThermoFisher Scientific).

### Example 10—Quantitative Image Analysis for RNAScope

Sections were imaged using a Leica DM6000 confocal microscope. Montage images of multiple 512×512 pixels were created and used for analysis. Follicular and extrafollicular areas were delineated using Leica software with B cell follicle areas identified morphologically as clusters of closely aggregated brightly stained CD20⁺ or IgM⁺ cells. Some sections were co-stained with Goat anti-human IgM-AF647 (Jackson ImmunoResearch) and mouse-anti-human CD20 antibodies (clone L26, Biocare Medical, Inc.) to confirm that both antibodies co-localized in B cell follicles similarly. Cell counts were done using LAS X (Leica confocal) software; each cell was demarcated using a Leica software tool to avoid counting the same cell twice. Leica software was used to measure the delineated areas for cell counts. To determine the percentage of follicles that has CAR/CXCR5 T-cells over time post infusion, a total of 790 follicles were evaluated for presence of CAR/CXCR5 T-cells with a median of 302 follicles per animal (range 172-316). To determine the levels of CAR/CXCR5 T-cells/mm² in follicular areas, over time post infusion, a median of 8.4 mm² (range 6.8-8.9 mm²) of follicular area was analyzed with a total of 172 follicles analyzed with a median of 57 follicles per animal (range 48-67). In addition, a total of 190 follicles were examined to determine the percentage of follicles that has a cluster of expanding CAR/CXCR5 T-cells at the edge of the follicle at 2 DPT with a median of 95 follicles (range 90-100). To determine the percentage of follicles with free virions bound by FDC over time post infusion, a total of 518 follicles with a median of 146 follicles per animal (range 140-232). To determine the levels of SIV RNA⁺ cells/mm² in follicular areas, over time post infusion, a median of 6.46 mm² (range 5.48-6.84 mm²) of follicular area was analyzed with a total of 131 follicles analyzed with a median of 45 follicles per animal (range 38-48). To determine levels of CAR/CXCR5- T-cells/mm² and level of SIV RNA⁺ cells/mm² in treated animals a median of 19.9 mm² per animal (range of 15.7-34 mm²) of EF areas was analyzed. To confirm the specificity of the custom made probe that we designed to detect the gammaretroviral CAR/CXCR5 construct and to determine the level of SIV vRNA⁺ cells in F areas, LN tissues from three untreated control animals were hybridized to the custom made probe and an SIV probe with a median of 2.36 mm² (range 0.79-2.99 mm²) of follicular area was analyzed with a total of 53 follicles analyzed with a median of 22 follicles per animal (range 5-26). To determine level of SIV RNA⁺Cells/mm² in untreated control animals a median of 3.6 mm² per animal (range of 0.68-9.3 mm²) of EF areas was analyzed.

### Example 11—Immunohistochemistry and Analysis

Indirect immunohistochemistry was performed on fresh tissue specimens shipped overnight, sectioned with a compresstome and stained essentially as previously described (Skinner et al., 2000, J. Immunol., 165:613-7; Li et al., 2017, J. Vis. Exp., 1-8; Abdelaal et al., 2019, Int. J. Mol. Sci., 20). Briefly, sections were stained with 0.4 μg/mL rabbit -anti-human CD20 polyclonal antibodies (Neomarkers) and 2 μg/mL rat-anti-human CD3 antibodies (clone MCA1477, BioRad). Then sections were stained with secondary antibodies by incubating with, 0.3 μg/mL Alexa Fluor 488-conjugated goat-anti-rabbit antibodies, and 0.2-0.3 μg/mL Cy5-conjugated goat anti-rat antibodies overnight at 4° C. Secondary antibodies were obtained from Jackson ImmunoResearch Laboratories (West Grove, Pa.). Sections were imaged using a Leica DM6000 confocal microscope. Montage images of multiple 512×512 pixels were created and used for analysis. Confocal z-series were collected in a step size of 3 μm. Images were opened and analyzed in LAS X (Leica confocal) software directly. We used the LAS X software to create montages of multiple projected confocal serial z-scans. Follicular areas were identified morphologically as clusters of brightly stained, closely aggregated CD20⁺ cells. Follicular and extrafollicular areas were delineated and measured using LAS X software. Areas were not included if they showed loosely aggregated B cells that were ambiguous. To prevent bias, the yellow CTV channel was turned off when follicular and extrafollicular areas were delineated. Cell counts were done on single z-scans.

### Example 12—Statistical Analysis

Statistical analysis utilized GraphPad Prism 8.3.0 for Windows (GraphPad Software, San Diego, Calif.). Specific tests are indicated in the Description of the Drawings. Correlations were determined using Spearman's correlation, assuming independence.

### Example 13—CAR/CXCR5 T-Cells Home to Lymphoid Follicles and Contact SIV-Infected Cells In Vivo

To evaluate the localization and relative abundance of CAR/CXCR5-T cells in lymphoid and non-lymphoid tissues, and the relative localization within lymphoid tissues of CAR/CXCR5 T-cells and SIV vRNA⁺ cells, we treated an SIVmac239-chronically infected rhesus macaque with autologous CAR/CXCR5-transduced T-cells and sacrificed the animal 2 days post-treatment (DPT). CAR/CXCR5 T-cells were labeled with the fluorescent dye Cell Trace Violet (CTV), and infused into the animal at a dose of 0.35×10⁸ cells/kg. Spleen, lymph node (LN), rectum, ileum, bone marrow, lung, liver, and brain were collected at 2 DPT, and examined for localization of the CTV-labeled cells (FIG. 1A). CAR/CXCR5 T-cells primarily accumulated in the B-cell follicles (F) and the extrafollicular T cell areas (EF) of the spleen and LN, with a few cells detected in the rectum and lung. The cells were not detected in ileum, bone marrow, liver, or brain tissue sections (FIG. 1A). To evaluate the localization of CAR/CXCR5 T-cells relative to SIV vRNA⁺ cells, we used duplex RNAScope in situ hybridization (ISH), which allows simultaneous detection of both gammaretroviral vector-transduced CAR/CXCR5 T-cells and SIV-infected cells. Spleen tissue sections were hybridized to two sets of probes, one that specifically binds the gammaretroviral CAR/CXCR5 construct and another that specifically binds SIV RNA (FIG. 1B). In addition to detecting SIV vRNA⁺ cells, SIV virions trapped by the follicular dendritic cells (FDC) network were detected as a white haze within the B-cell follicle as previously described. The duplex RNAScope ISH was combined with immunofluorescence staining to allow the delineation of F and EF areas of SLT. We found CAR/CXCR5 T-cells were primarily detected in F areas of the spleen (20.1 cells/mm² in F compared to 3.8 in EF) and, in some instances, were detected in direct contact with SIV vRNA⁺ cells. We found 4% (23/621) of SIV vRNA⁺ cells were in direct contact with CAR/CXCR5 T-cells.

### Example 14—Infusion of CAR/CXCR5 T-Cells into SIV-Infected Rhesus macaques is Safe

We next investigated the safety and in vivo efficacy of CAR/CXCR5 T-cell immunotherapy in SIV-infected ART suppressed animals, compared to untreated control animals. The untreated and treated animal groups included male and female animals, of similar age, weight, peak viral loads and CD4/CD8 frequencies (Table 2).

TABLE 2
Animal Information

| | | | | | Peak VL | Pre-ART | ART | |
|---|---|---|---|---|---|---|---|---|
| | | Age | | Weight | Post- | CD4/CD8 | treatment | Necropsy |
| Group | Animal | (yrs) | Gender | (kg) | infection | frequency | (days) | (DPT) |
| | R14025 | 3.6 | M | 5.5 | 3.80 × 10⁷ | — | — | 2 |
| T1 | R01093 | 17 | F | 7.6 | 0.94 × 10⁷ | 0.44 | 337 | 56 |
| T1 | Rh2526 | 9.8 | F | 5.0 | 4.74 × 10⁷ | 0.76 | 379 | 70 |
| T1 | Rh2537 | 13 | F | 8.0 | 0.94 × 10⁷ | 1.28 | 358 | 83 |
| C | R12049 | 6.3 | F | 6.4 | 6.37 × 10⁷ | 1.06 | 337 | 56 |
| C | R11100 | 7.1 | F | 7.4 | 4.62 × 10⁷ | 0.31 | 379 | 70 |
| C | R10002 | 9 | M | 11.2 | 2.75 × 10⁷ | 0.77 | 358 | 83 |
| T2 | Rh2850 | 4.7 | M | 6.3 | 0.93 × 10⁷ | 1.08 | 104 | 308 |
| T2 | Rh2853 | 7.1 | M | 9.2 | 2.00 × 10⁷ | 1.29 | 76 | 77 |
| T2 | Rh2858 | 6.4 | M | 10.6 | 2.38 × 10⁷ | 0.68 | 90 | 328 |

In the first group of treated rhesus macaques (T1), peripheral blood mononuclear cells (PBMCs) for CAR/CXCR5 transduction were collected during the chronic stage of infection.

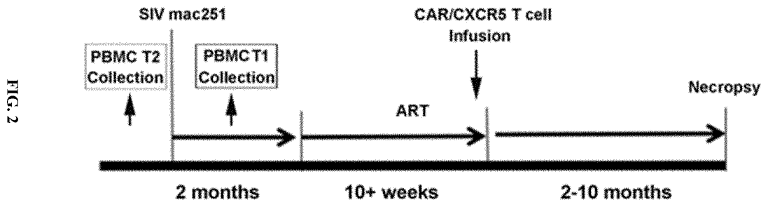

For the second treatment group (T2), PBMCs for CAR/CXCR5 transduction were collected prior to SIVmac251 infection. T1 and T2 animals, as well as control animals were suppressed with antiretroviral therapy (ART) that was initiated 63-68 days post-infection. Animals were released from ART at the time of CAR/CXCR5 T cell infusion and monitored for at least 60 days as outlined in the study design shown in FIG. 2. Blood and tissue samples were collected over time to monitor infused cells and SIV viral RNA.

To evaluate that safety of the treatment, animals were monitored by veterinary staff twice daily for any signs of pain, illness, and stress by observing appetite, stool, behavior, and physical condition in response to the infused CAR/CXCR5 T-cells. The animals exhibited no observable adverse ill effects after receiving the immunotherapeutic cells and their weights were unaffected by the immunotherapeutic infusion. Necropsy reports noted no abnormalities in treated animals beyond those typical in SIV-infected animals.

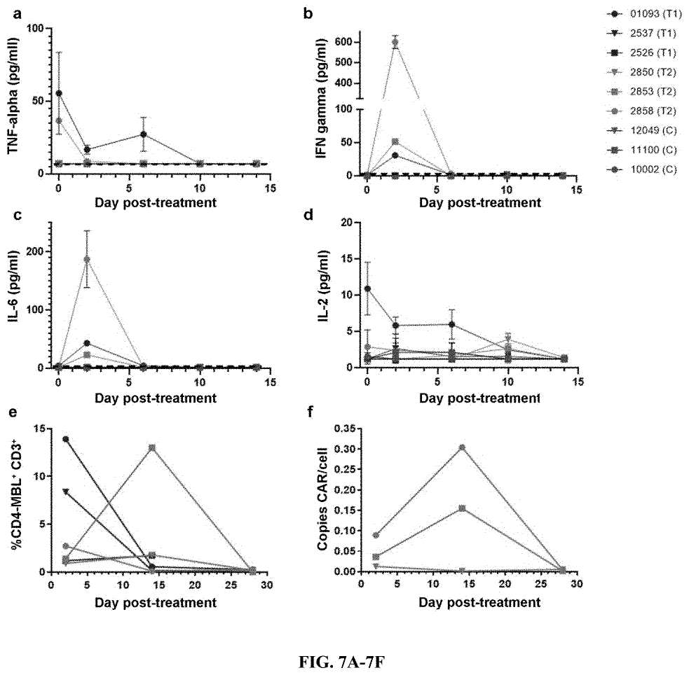

A Luminex assay, for monitoring cytokine levels after the cell infusion, showed a transient spike in IL-6 and interferon gamma (IFN-gamma) at 2 DPT in three of the six treated animals; and levels returned to normal by 6 DPT (FIG. 7A-7D). The CAR CXCR5 T-cells did not accumulate in lung tissues (FIG. 1); however, we were able to detect cells in bronchoalveolar lavage (BAL) samples shortly after infusion by flow cytometry (FIG. 7E) and quantitative polymerase chain reaction (qPCR) (FIG. 7F). The cells likely accumulated in the BAL shortly after infusion due to pulmonary circulation. Upon necropsy, the lungs appeared healthy. The overall health of the animals and the transient nature of the cytokine spikes suggests that the infusion of autologous CAR/CXCR5 T-cells is safe.

### Example 15—Prior to Infusion, CAR/CXCR5 Cells were Predominantly Activated Central Memory T Cells

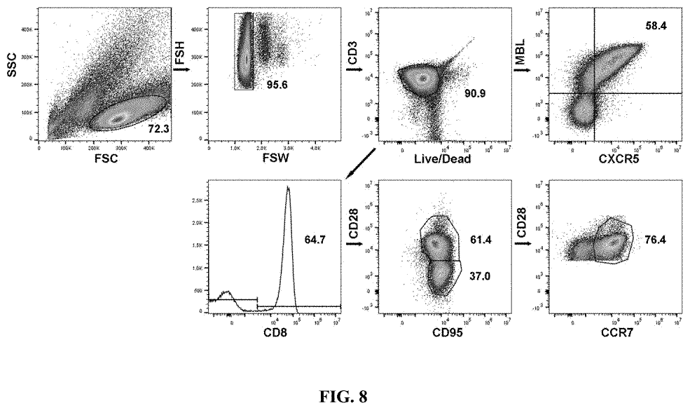

The dose of CAR/CXCR5 transduced T-cells infused into T1 and T2 animals ranged from 0.8-2.0×10⁸ cells/kg (Table 3). The infused cells were a mix of CD8 and CD4 T-cells. Most of the infused cells expressed both the CAR and CXCR5 (range, 55-79.4%) and primarily displayed a central memory phenotype (range, 50.3-73.6%) (Table 3, FIG. 8). The majority of the central memory cells expressed C-C chemokine receptor 7 (CCR7) (range, 44.6-93.5%), a lymph node homing molecule.

TABLE 3
Infusion cells

| Group | Animal | Cells/kg infused | % CAR/CXCR5 | % CM | % CCR7+ | % CD4⁺ | % CD8⁺ | % CD4⁺CD8⁺ |
|---|---|---|---|---|---|---|---|---|
| T0 | R14025 | 0.35 × 10⁸ | 66.2 | 58.1 | 93.5 | 43.8 | 16.3 | 38.0 |
| T1 | R01093 | 0.8 × 10⁸ | 55.0 | 50.3 | 66.5 | 22.3 | 46.5 | 28.8 |
| T1 | Rh2526 | 1.12 × 10⁸ | 59.5 | 65.3 | 62.5 | 26.8 | 36.0 | 35.4 |
| T1 | Rh2537 | 0.75 × 10⁸ | 57.4 | 66.9 | 59.0 | 42.3 | 27.3 | 28.9 |
| T2 | Rh2850 | 2.0 × 10⁸ | 79.4 | 73.6 | 62.5 | 20.6 | 14.3 | 63.8 |
| T2 | Rh2853 | 1.06 × 10⁸ | 68.6 | 54.5 | 44.6 | 32.3 | 15.3 | 48.3 |
| T2 | Rh2858 | 1.26 × 10⁸ | 58.4 | 61.4 | 76.4 | 33.4 | 23.2 | 41.9 |

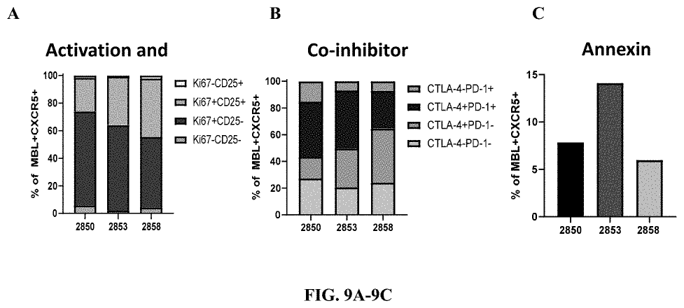

To gain a more complete understanding of the pre-infusion phenotype of CAR/CXCR5 transduced cells, we conducted a more in depth phenotypic and functional analysis of the CAR/CXR5 T cells that were infused into the T2 animals. In cells produced from all three animals, over 90% were activated and proliferating as measured by Ki67 and 25-45% of cells were activated as measured by CD25 expression (FIG. 9A). There was a variable expression of the inhibitory markers PD-1 and CTLA-4, but co-expression of these inhibitory markers was found on less than 50% of transduced cells (FIG. 9B). We also examined the frequency of cells undergoing apoptosis and found that overall apoptosis levels were low (FIG. 9C). However, at the time of infusion, cells from one of the animals (Rh2853), were undergoing apoptosis at 1.8-2.4 times the frequency of cells of the other two animals (Rh 2850, Rh2858) (FIG. 9C). Taken together, this analysis suggests that at infusion, the transduced cells were of primarily central memory phenotype, were activated, proliferating, and viable.

### Example 16—CAR/CXCR5 T Cell Infusion Associated with Reductions in Viral Loads

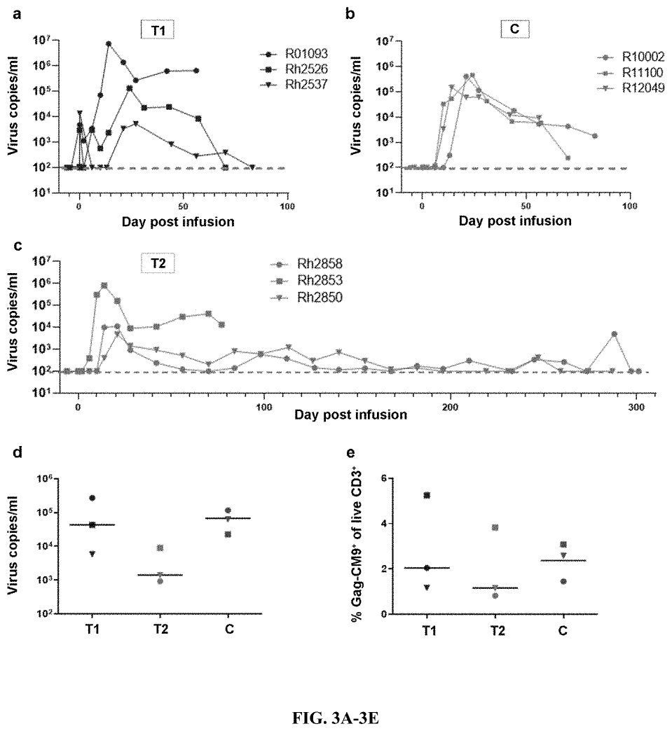

After infusion of CAR/CXCR5 cells into rhesus macaques, viral loads were monitored. Untreated control animals (FIG. 3A) showed a rapid rise in viral loads after ART cessation, followed by a slow decline over time that never reached undetectable levels. T1 animals (FIG. 3B), in which cells were collected during chronic untreated infection, showed an initial spike in viral loads due to the presence of virus in the infused SIV-infected transduced cells. Viral loads dropped in all three treated animals (to undetectable in 2/3 ) shortly after infusion, and then began to rise. One of the three T1 treated animals maintained substantially lower viral loads compared to control untreated animals for the duration of the experiment, and 2/3 of treated animals had undetectable viral loads at necropsy. In T2 animals (FIG. 3C), 2/3 of treated animals showed lower peak viral loads post-ART release compared to control untreated animals. One-month post-infusion (27-30 DPI), the viral loads in 2/3 T1 and 3/3 T2 animals were lower than untreated control animals, with the median viral load of T2 being nearly 2 logs below that of the untreated control animals (FIG. 3D). As an exception to the study design, which was planned to maintain animals for only 2-3 months post-cell infusion and ART release, we maintained two T2 animals for 10 months post-infusion in order to monitor long-term viral loads. During this time, the animals-maintained control of infection, with viral loads oscillating between undetectable and very low levels (FIG. 3D). In addition, we examined and detected similar levels of naturally occurring SIV-specific (Mamu-A1*001/Gag-CM9) CD8⁺ T-cells in PBMCs of treated and untreated control animals at one-month post-infusion (FIG. 2E). This finding suggests that the differences in viral loads between groups was not driven by differences in the endogenous response. Overall, these data suggest that CAR/CXCR5 T-cell therapy is effective at reducing viral loads in SIV-infected rhesus macaques after ART cessation.

### Example 17—CAR/CXCR5 T-Cells Expand In Vivo

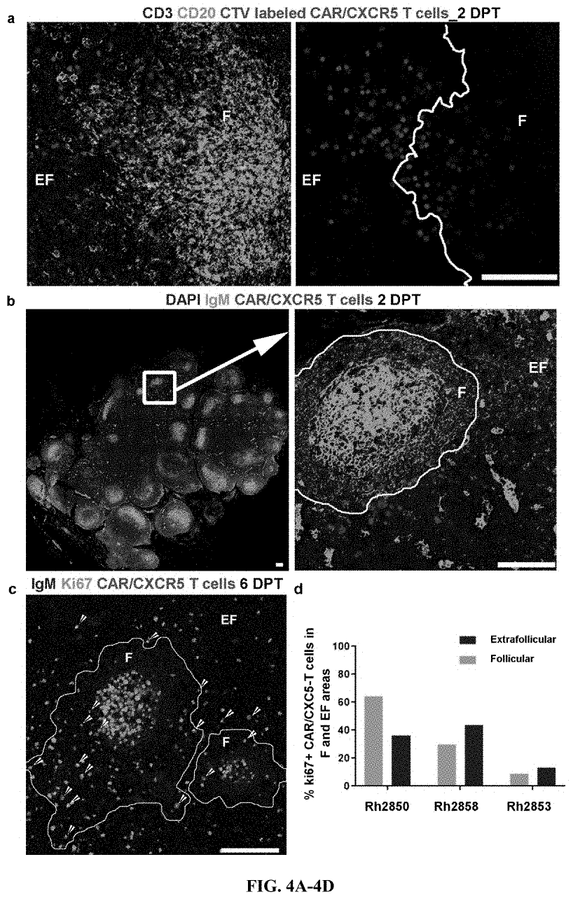

At 2 DPT, CTV-labeled CAR/CXCR5-transduced cells showed evidence of proliferation in both the EF and F areas of lymph nodes, showing doublets of cells and cells with decreased fluorescent intensity indicating a loss of CTV with cell division. Cells within the F areas had an overall lower CTV fluorescence intensity than those in the EF (FIG. 4A), suggesting that they had undergone further cell division. Similarly, at 2 DPT, RNAScope detection of CAR/CXCR5 T-cells combined with immunofluorescent staining of lymph nodes showed clusters of CAR/CXCR5 T-cells at the edge of the follicles, suggestive of cell expansion (FIG. 4B). These clusters were detected in over 50% of the follicles (range 49-58%). In vivo proliferation of CAR/CXCR5 T-cells was further confirmed at 6 DPT in lymph node sections in treated animals by a combination of RNAScope and Ki67 antibody staining to mark T-cell activation and proliferation. We detected Ki67⁺CAR/CXCR5 T-cells, in both F and EF areas (FIG. 4C). Levels of Ki67⁺CAR/CXCR5 T-cells ranged from (9-64%) with a median of 30% of the total CAR/CXCR5 T-cells in F areas and ranged from (13-44%) with a median of 36% of total CAR/CXCR5 T-cells in EF areas. Importantly, the T2 animal demonstrating the greatest control (Rh2850), showed the highest percentage of F Ki67⁺CAR/CXCR5 T-cells (64%) and the animal that lost control (Rh2853) showed the lowest percentage of follicular Ki67⁺CAR/CXCR5 T-cells (9%) (FIG. 4D).

### Example 18—CAR/CXCR5 T-Cells Localize to the Follicle and Persist for up to 28 Days

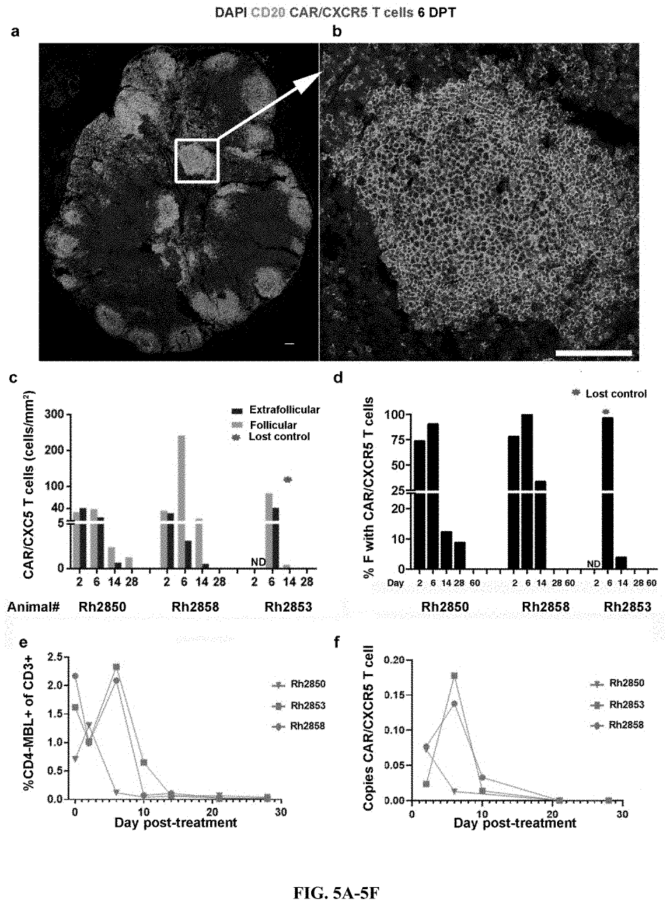

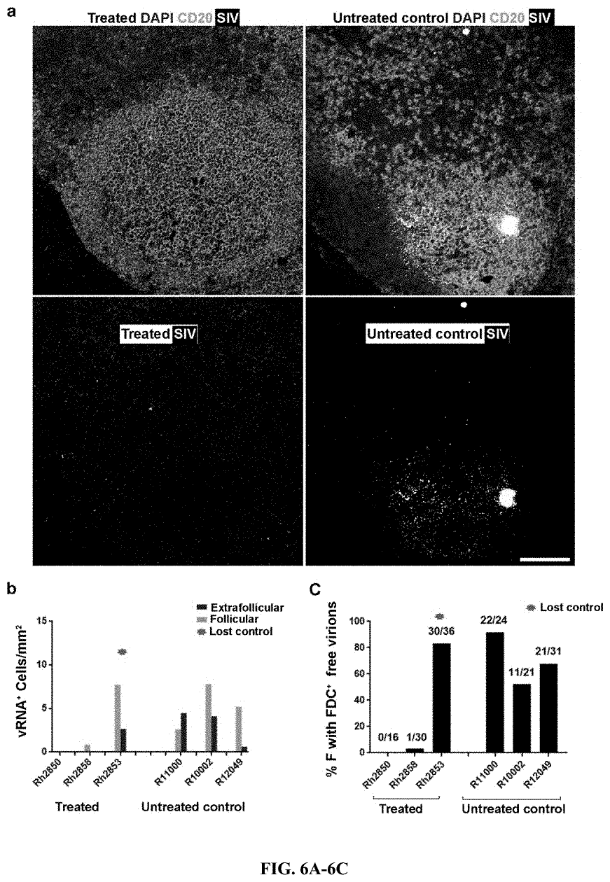

We analyzed CAR/CXCR5 T-cells in sections of LN from T2 animals biopsied at 2, 6, 14, 28, and 60 days post-treatment using RNAscope. There was a noticeable shift at 6 DPT to CAR/CXCR5 T-cells primarily accumulating within B-cell follicles (FIG. 5B compared to FIG. 4B). We quantified CAR/CXCR5 T-cells in the F and EF regions of LNs. CAR/CXCR5 T-cells were most abundant during the first week post-infusion, followed by a decline over time (FIG. 5C). At 2 DPT, CAR/CXCR5 T-cells were detected at similar levels in both F and EF areas, with a median of 28 cells/mm² (range, 26-30) in F areas and 30 cells/mm² (range, 23-37) in EF areas (FIG. 6C). At 6 DPT the cells were detected predominantly in F areas with a median of 78 cells/mm² (range, 34-239) compared to a median of 11 cells/mm² (range, 3-38) in EF areas. (FIG. 6C). At 14 DPT, the F:EF ratio increased, however, the overall frequency of cells sharply declined in all of the treated animals, with a median of 2.3 cells/mm² (range, 0.32-7.6) in F and 0.4 cells/mm² (range, 0-0.57) in EF areas (FIG. 6C). By 28 DPT, cells were only detected in F areas of one animal (Rh2850; 1.17 cells/mm²), with no cells detected at 60 DPT in any of the examined sections of the treated animals (FIG. 6C). Notably, the animal that lost viral control (Rh2853) showed the fastest and steepest decline in levels of CAR/CXCR5 T-cells over time relative to two animals that controlled infection.

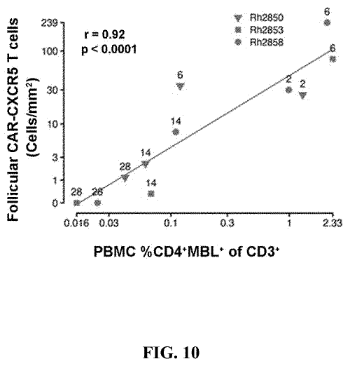

We also determined the percentage of follicles in LNs that contained CAR/CXCR5 T-cells. During the first week post-treatment, most follicles had detectable CAR/CXCR5 T-cells. In fact, at 6 DPT; a median of 96% (range, 90-100%) of follicles examined had CAR/CXCR5 T-cells (FIG. 4D). These levels declined in all animals at subsequent time points. Examination of PBMCs using both flow cytometry and qPCR revealed a similar pattern of CAR/CXCR5 T-cell persistence. CAR T-cells were detectable in isolated PBMCs up to 14-21 DPT by flow cytometry (FIG. 7E). Genomic DNA PCR detection of CAR/CXCR5 T-cells in PBMCs showed a similar decline in cell number by day 14 (FIG. 7F). In addition, we found a strong positive correlation between levels of follicular CAR/CXCR5 T-cells in LN tissue in situ and the frequency of CD4-MBL⁺ CAR cells detected in PBMCs by flow cytometry (FIG. 10). This finding suggests that the cells have similar persistence in peripheral blood and tissue.

### Example 19—In Vivo Levels of Viral RNA Appear to be Impacted by CAR/CXCR5 T-Cell Infusion

We determined the levels of vRNA in the three T2 animals and three control animals at 28 DPT (FIG. 6). The two treated animals that exhibited sustained control of SIV infection (Rh2850, Rh2858) showed few to no SIV vRNA⁺ cells at 28 DPT in F and EF areas compared to abundant SIV vRNA⁺ cells in untreated control animals and the T2 animal that did not control the infection (Rh2853) (FIGS. 6a and 6b). In addition, Rh2850 and Rh2858 animals had lower percentages of follicles with free virions trapped by the FDC network than untreated control animals, or the treated animal that lost control (Rh2853) (FIG. 6c). In fact, Rh2850 had no detectible FDC associated virions in any follicles, and only 1 of 30 follicles showed FDC associated virions in Rh2858, whereas most follicles showed FDC trapped virions in untreated control animals, and the treated animal that lost control. These findings suggest that the immunotherapeutic cells may have led to sustained reductions in vRNA in the treated animals. In addition, we detected no CAR/CXCR5 T-cells that were SIV vRNA⁺ in the examined sections.

### Example 20—Procedures Using Pre-Treatment with Anti-CD20 Antibody in Rhesus macaques

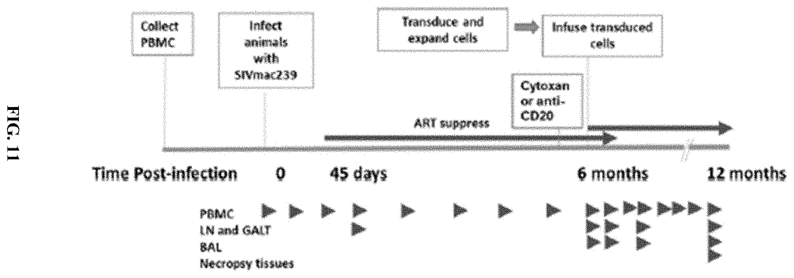

Animals and SIVmac239 infection: Infections of up to 26 unrelated male and female Indian Rhesus macaques (Macaca mulatta) with SIVmac239 are performed at the WNPRC. Most Rhesus macaques for this study are positive for the Mamu-A*001:01 MHC-I allele. Mamu-A*001:01 macaques develop robust well-defined CD8 T cells responses and exhibit a disease similar to HIV6,8,91, and develop viral rebound after release from suppressive antiretroviral therapy. Animals that possess MHC-I alleles associated with spontaneous control of viremia, e.g., B*08 or B*1792,93, are excluded from the study. SIV infections are performed intrarectally with 1000-3000 TCID50 of SIVmac239 virus stock. These studies include 4 groups of SIV infected ART suppressed animals (FIG. 11):
Group 1) animals treated with autologous CAR/CXCR5-transduced CD8 T cells (n=7)
Group 2) a control group that is not treated with gene modified cells (n=7)
Group 3) animals pre-treated with anti-CD20 and infused with CAR/CXCR5 T cells (n=6)
Group 4) a control group that is pre-treated with anti-CD20 but not treated with gene modified cells (n=6)

Blood Draws: Animals undergo repeated blood draws prior to infection to obtain

PBMCs for transduction. After SIVmac239 infection, blood samples are collected to monitor plasma viral burden, and the potential development of anti-CAR responses, as well as the quantity of key CD4+ and CD8+ T cell subpopulations (naive, memory, regulatory subsets), the transduced CAR/CXCR5 T cells, tetramer positive SIVmac239-specifc T cells, NK cells, and B cells.

Anti-Viral Therapy: At day 35-45 post-infection, animals initiate a potent antiviral therapy consisting of dolutegravir, tenofovir, and emtricitabine administered once daily subcutaneously. This regimen has been shown to lead to virologic suppression (<50 SIV RNA copies/ml) in SIV-infected rhesus macaques within 2 to 3 months. Plasma viral load is monitored every two weeks at the WNPRC using an assay that has been adapted for sensitivity down to >100 copies SIV RNA/ml.

Immunodepleting Preconditioning: Pretreatment by temporarily depleting B cells is carried out as previously described (Schmitz et al., 2003, J. Virol., 77:2165-73; Li et al., 2016, J. Virol., 90:11168-80) using the rhesus recombinant depleting anti-CD20 antibody similar to or derived from Rituximab (human anti-CD20 used in the clinic) supplied by the

NIH Non-Human Primate Reagent Resource (i.e., anti-CD20 [anti-CD20 (2B8R1), Nonhuman Primate Reagent Resource Cat #PR-2287, RRID: AB_2716323] derived from Rituximab and grafted into rhesus variable regions).

Therapeutic T Cell Infusions: Six months after initiation of ART, and one week after pre-treatment, animals receive therapeutic T cell infusions, assuming that they have achieved and maintained undetectable viral loads. Therapeutic CD4-MBL-CAR/CXCR5 are placed in media containing 10% autologous serum and transported to the WNPRC for infusion. Each treated animal is paired with an untreated negative control animal. Immediately prior to infusion, a small aliquot of cells is removed and stained with antibodies to determine the percentage of CAR T cells, and their memory, regulatory, and activation phenotypes, as described herein. Cells are infused intravenously at a dose of 1 to 2×10\hat{ }8 cells/kg.

Tissue Processing: Lymphoid tissues (LN, GALT) obtained prior to necropsy are partitioned and half fixed in 4% paraformaldehyde for RNAscope and DNAscope studies and half snap-frozen in OCT for immunostaining to determine localization of CAR T cells. BAL is centrifuged, cells are cryopreserved and stained by flow cytometry to determine frequencies and phenotypes of transduced T cells. At necropsy, LN (inguinal, axillary, mesenteric), spleen, jejunum, rectum, CSF, brain, nasopharyngeal tissue, liver, lung, genital tract tissues including vagina, cervix, and uterus in females, and testes, vas deferens, and epididymis in males are collected and processed as follows. Portions of each tissue are 1) fixed in 4% paraformaldehyde and paraffin embedded; 2) snap frozen and OCT embedded, 3) fresh, placed in chilled RPMI, for immunostaining studies to localize and quantify CAR/CXCR5 T cells; and 4) for spleen and LN only, immediately disaggregated by conventional techniques at the WNPRC, cryopreserved, stored in liquid nitrogen and later used to analyze the frequency and phenotype of CAR/CXCR5 T cells.

### Example 21—Pre-Treatment with CD20 Antibodies Improves the Abundance, Persistence, or Antiviral Efficacy of Autologous CD4-MBL-CAR/CXCR5-Transduced CD8 T Cells Infused into ART-Suppressed Rhesus macaques

Temporary depletion of B cells temporarily disrupts B cell follicles and the follicular viral reservoir and creates space to allow for homeostatic proliferation of infused autologous CD4-MBLCAR/CXCR5 T cells.

Temporary B cell depletion shows similar or increased levels of CD4-MBLCAR/CXCR5 T cell localization and persistence, compared to animals that did not undergo this pre-treatment.

Temporary B cell depletion shows similar or superior suppression of viral replication by CD4-MBL-CAR/CXCR5 T cells compared to animals that did not receive CD20 antibody pre-treatment.

While immunodepletion pretreatment with cyclophosphamide is state-of-the-art for current immunotherapy approaches, it is quite toxic and introduces risks to an immunotherapy approach for an HIV cure strategy. An alternative pretreatment approach that is much less toxic and may produce superior results for HIV cure strategies, including CD4-MBL-CAR/CXCR5 T cell therapy, is temporary depletion of CD20+ cells. CD20 cell depletion with Rituximab has been shown to be safe and effective in humans. A rhesus version of Rituximab has been produced and is safe and effective in rhesus macaques⁹⁴. The rationale for this approach is based on HIV and SIV concentrating in B cell follicles, both as virions accumulating on FDC, and replicating cells. Depletion of B cells with anti-CD20 antibodies will temporarily disrupt B cell follicles and expose associated virions and viral replicating cells to incoming immunotherapeutic cells, while leaving other immune cells intact. It will also, at the same time, create space in lymph nodes to allow for and promote homeostatic proliferation and engraftment of immunotherapeutic cells. Thus, preconditioning with temporary depletion of CD20 cells for HIV cellular immunotherapy approaches offers a safe and potentially superior approach to conventional cyclophosphamide preconditioning.

The localization, persistence, and antiviral effects of autologous CAR/CXCR5-transduced CD8 T cells infused into ART suppressed, anti-CD20 pre-treated, SIV-infected animals is evaluated before and after treatment interruption. Results are compared to untreated animals, animals infused with CAR/CXCR5 T cells that were not pre-treated with CD20 depleting antibodies, and animals pretreated with CD20 depleting antibodies with no CAR/CXCR5 T cell infusion.

### Sample Size Calculations and Data Analysis

All power calculations were conducted, and all data analyses will be conducted, in R (at rproject.org/ on the World Wide Web). Given small sample sizes and potentially skewed outcomes, non-parametric tests (Wilcoxon rank-sum test) and summary statistics (median/range) are used as appropriate. A sample size of 6 animals per group provides 80% (90%) power to detect a difference if the probability of superiority is at least 91.5% (94.3%), using a Wilcoxon rank-sum test with a significance level of 0.05; if normally distributed, this is an effect size (mean/SD) of 1.94 (2.23).

The primary endpoint is a comparison of frequencies of transduced T cells in B cell follicles between animals pre-treated with CD20 depleting antibodies infused with CAR/CXCR5 T cells compared to untreated animals, animals infused with CAR/CXCR5 T cells that were not pretreated with CD20 depleting antibodies, and animals pretreated with CD20 depleting antibodies with no CAR/CXCR5 T cell infusion in lymph nodes and spleen 1 month post-infusion. Secondary endpoints are comparison of the treated groups with regard to frequencies of CD4-MBL-CAR/CXCR5 T cells in B cell follicles of lymph nodes and GALT at days 2, 10, 28 post-infusion and at the end of the experiment; frequencies in the blood; and the follicular:extrafollicular (F:EF) ratio of CD4-MBL-CAR/CXCR5 T cells compared to control animals in lymph nodes and spleen.

The primary endpoint is comparison of plasma viral load between animals pre-treated with CD20 depleting antibodies infused with CAR/CXCR5 T cells compared to untreated animals, animals infused with CAR/CXCR5 T cells that were not pretreated with CD20 depleting antibodies, and animals pretreated with CD20 depleting antibodies with no CAR/CXCR5 T cell infusion. Secondary endpoints include time to viral rebound, peak viremia, viral load at the end of the experiment, frequencies of SIV RNA+ and DNA+ cells in follicular and extrafollicular regions of lymphoid tissues, F:EF ratio of vRNA+ and vDNA+ cells in tissues, as well as amount of FDC-bound vRNA in lymph node and spleen.

### Interpretation

It is likely that animals pre-treated with CD20 depleting antibodies compared to control-treated animals show greater frequencies of transduced T cells in B cell follicles in lymph nodes and spleen 1 month post-infusion. It is also likely that the CD20 depleted and CAR/CXCR5 treated group shows greater follicular:extrafollicular (F:EF) ratios in lymph nodes at spleen 6 months post-infusion, and similar or greater frequencies of CD4-MBLCAR/CXCR5 T cells in B cell follicles of lymph nodes and GALT (days 2, 10, 28).

It is possible that a delay in time to viral rebound and decreased viral loads animals pretreated with CD20 depleting antibodies compared to the control groups is observed. It also is possible that reductions in peak viremia, frequencies of SIV RNA+ and DNA+ cells in follicular and extrafollicular regions of lymphoid tissues, F:EF ratio of vRNA+ and vDNA+ cells in tissues, as well as amount of FDC-bound vRNA in lymph node and spleen are observed. It is likely that similar or increased delays in viral rebound and decreased viral loads compared to control animals are observed.

Collectively, these findings support using temporary CD20 depletion as a safe alternative cyclophosphamide for HIV cure immunotherapy approaches.

### Example 22—Anti-CD20 Antibody Pre-Treatment Led to a Reduction of B Cells and Follicles in Lymphoid Tissues

An SIV-infected, ART-suppressed rhesus macaque was treated with 7 mg/kg anti-CD20 antibody seven days prior to ART release and the infusion of CAR/CXCR5 T cells. The animal was sacrificed 2 days post-infusion (9 days post-B cell depletion) and levels of B cells were evaluated in situ using immunohistochemistry. In spleen sections, no B cells or follicles were detected. In lymph node sections, B cells and follicles were detected, however the overall levels of B cells were greatly reduced and the follicles fewer and smaller than untreated animals. See FIG. 12.

### Example 23—Anti-CD20 Antibody Pre-Treatment Led to a Temporary Reduction of B Cells in Blood

SIV-infected, ART-suppressed rhesus macaques were treated with 7 mg/kg anti-CD20 antibodies seven days prior to ART release and the infusion of CAR/CXCR5 T cells. The measurement of CD20+cells in the blood over time after anti-CD20 antibody treatment by flow cytometry indicates that CD20+ cells were depleted and gradually recovered. The CD20+ cells returned to normal levels after 2 months. See FIG. 13.

This method of use of CD20 depleting antibodies is unique. It is lower than the normal prescribed use of the drug to deliberately make the depletion near complete but very short. The prescribed dose of anti-CD20 antibodies (e.g., Rituximab) for treating cancer and autoimmune diseases ranges from 9.6-13.5 mg/kg per dose, and at least 2 to 8 or more doses (see Table 1). We show herein that a single dose of 7 mg/kg was sufficient to temporarily deplete B cells from the blood and secondary lymphoid tissues with normal B cell levels returning in 2 months.

### Example 24—In Animals Pre-Treated with Anti-CD20 Antibody, CAR T Cells Expanded in Lymph Nodes During the First Week Post-Infusion in the Absence of Detectible Antigen (viral RNA) in the Same Lymph Nodes

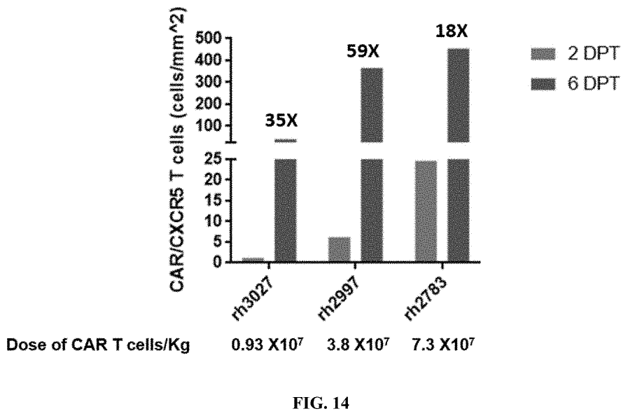

SIV-infected, ART-suppressed rhesus macaques were treated with 7 mg/kg anti-CD20 antibody (derived from Rituximab) seven days prior to ART release and the infusion of CAR/CXCR5 T cells. Increasing doses of CART cells were infused. Doses are indicated at the bottom of the figure and ranged from 0.93×10⁷ to 7.3×10⁷ cells/kg. The concentration of CAR T cells and SIV vRNA+ was determined at days 2 and 6 post-infusion in lymph nodes in situ using RNAscope analysis. No vRNA+ cells were detected. The concentrations of CAR/CXCR5 T cells and the fold increase of cells from 2 to 6 days post-infusion was determined. Levels of CAR/CXCR5 T cells were 18-59 times greater in lymph nodes at 6 weeks post-infusion compared to 2 weeks post-infusion. See FIG. 14. These data suggest that the CAR T cells underwent robust expansion during the first week post-infusion in animals that were temporarily depleted of B cells.

### Example 25—After Anti-CD20 Antibody Pre-Treatment, CAR/CXCR5 T Cells Accumulated to Extremely High Levels in Follicular Areas of Lymph Nodes 6 Days Post-Infusion

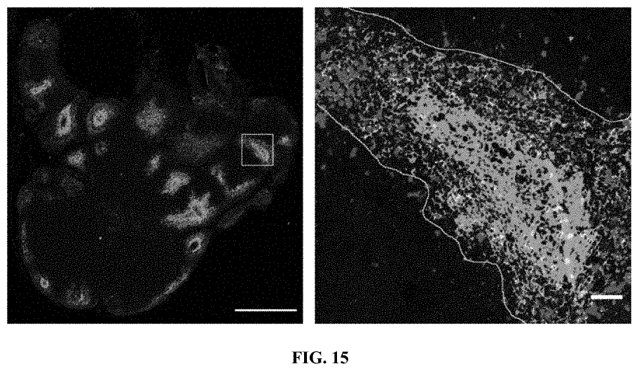

CAR/CXCR5 T cells were not evenly distributed in lymph node tissues, but preferentially accumulated in lymph node follicles. This is presumably due to the expression of the CXCR5 chemokine receptor on the CAR T cells. CAR/CXCR5 T cells reached extremely high levels of 900 and over 1000 cells/mm² in lymph node follicle biopsies collected 6 days post-infusion from animals rh2783 and rh2997, respectfully. See FIG. 15.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of temporarily depleting CD20+ cells in an individual being treated for HIV, comprising:
administering CD20+ cell-depleting therapy to the individual for a period of time prior to and/or while the individual is receiving anti-HIV therapy,
thereby temporarily depleting CD20+ cells in the individual.

2. The method of claim 1, further comprising administering the anti-HIV therapy.

3. The method of claim 1, wherein the CD20+ cell-depleting therapy comprises an anti-CD20 antibody.

4. The method of claim 3, wherein the anti-CD20 antibody is selected from Rituximab and variants thereof, Ofatumumab, Obinutuzumab, Ibritumomab tiuxetan, and Ibritumomab tiuxetan.

5. The method of claim 1, wherein the CD20+ cell-depleting therapy is administered intravenously or intraparentally.

6. The method of claim 1, wherein the CD20+ cell-depleting therapy is administered at a dose of about 3 mg/kg recipient body weight up to about 9 mg/kg recipient body weight.

7. The method of claim 1, wherein the period of time is from about 1 week to about 2 weeks prior to the individual receiving the anti-HIV therapy.

8. The method of claim 1, wherein the anti-HIV therapy is selected from anti-HIV immunotherapy, “shock and kill” therapy strategies, anti-viral therapy (AVT), and stem cell therapy.

9. The method of claim 8, wherein the anti-HIV immunotherapy is selected from NK cell therapy, autologous T cell therapy, and allogeneic T cell therapy.

10. The method of claim 1, wherein the anti-HIV therapy comprises CAR/CXCR5 T cell therapy.

11. The method of claim 10, wherein the CAR/CXCR5 T cell therapy is administered after the administration of the CD20+ cell-depleting therapy.

12. The method of claim 1, wherein administering the CD20+cell-depleting therapy disrupts B cell follicles.

13. The method of claim 1, wherein the CD20+ cells that are temporarily depleted are mature B cells in B cell follicles.

14. A method of preconditioning an individual for anti-HIV therapy, comprising:
administering CD20+ cell-depleting therapy to the individual for a period of time prior to the individual receiving the anti-HIV therapy.

15. The method of claim 14, further comprising administering the anti-HIV therapy.

16. The method of claim 14, wherein the CD20+ cell-depleting therapy comprises an anti-CD20 antibody.

17. The method of claim 16, wherein the anti-CD20 antibody is selected from Rituximab, Rituximab and variants thereof, Ofatumumab, Obinutuzumab, Ibritumomab tiuxetan, and Ibritumomab tiuxetan.

18. The method of claim 14, wherein the CD20+ cell-depleting therapy is administered intravenously or intraparentally.

19. The method of claim 14, wherein the CD20+ cell-depleting therapy is administered at a dose of about 3 mg/kg recipient body weight up to about 9 mg/kg recipient body weight.

20. The method of claim 14, wherein the period of time is from about 1 week to about 2 weeks prior to the individual receiving anti-HIV therapy.
